(19) United States
(12) Patent Application Publication
(10) Pub. No.: US 2021/0355501 A1
(43) Pub. Date: Nov. 18, 2021

(54) INTERLEUKIN-8 FOR MAINTENANCE OF HUMAN ACUTE MYELOID LEUKEMIA AND MYELODYSPLASTIC SYNDROME AND USES THEREOF

(71) Applicant: Albert Einstein College of Medicine, Bronx, NY (US)

(72) Inventors: Amit Verma, Bronxville, NY (US); Ulrich Steidl, New Rochelle, NY (US); Britta Will, Armonk, NY (US); Tihomira Todorova, New Hyde Park, NY (US)

(21) Appl. No.: 17/278,439

(22) PCT Filed: Sep. 23, 2019
(86) PCT No.: PCT/US19/52348

§ 371 Date: Mar. 22, 2021

Related U.S. Application Data
(60) Provisional application No. 62/735,213, filed on Sep. 24, 2018.

(51) Int. Cl. C12N 15/85; A01K 67/027

(52) U.S. Cl. CPC C12N 15/8509; A01K 67/0275; A01K 2267/0331; A01K 2217/203; A01K 2227/105

(57) ABSTRACT

Methods are disclosed for enhancing growth of a human acute myeloid leukemia (AML) sample, a human myelodysplastic syndrome (MDS) sample, a human IL-8 dependent tumor sample, human preleukemia cells, and a human preleukemia clone or subclone ex vivo or in a xenograft animal model comprising adding human interleukin-8 (hIL-8) or a hIL-8 agonist to the sample or administering hIL-8 or a hIL-8 agonist to the animal model or expressing a gene encoding hIL-8 or a hIL-8 agonist in the animal model. The invention also provides a transgenic animal that expresses a gene encoding human interleukin-8 (hIL-8) or a hIL-8 agonist, which can be used to test the effectiveness of treatments for AML, MDS and IL-8 dependent tumors.

### BACKGROUND OF THE INVENTION

Throughout this application various publications are referred to in parentheses. Full citations for these references may be found at the end of the specification. The disclosures of these publications are hereby incorporated by reference in their entirety into the subject application to more fully describe the art to which the subject invention pertains.

Acute myeloid leukemia (AML) and myelodysplastic syndromes (MDS) are heterogeneous clonal neoplastic diseases that originate from transformed cells that have progressively acquired critical genetic changes that disrupt key differentiation- and growth-regulatory pathways (Hanahan and Weinberg 2000). Recent experimental evidence suggests that AML and MDS originate from early hematopoietic stem cells (HSCs) following the acquisition of multiple genetic or epigenetic changes that initially give rise to pre-leukemic HSC (pre-LSC) and then to fully transformed leukemia stem cells (LSC). Relapse continues to be the major cause of death in most subtypes of AML, suggesting that current therapies are largely ineffective in eliminating LSC and pre-LSC. As a consequence, future treatments should not only aim at reducing the bulk tumor (blast) population but must be directed against pre-LSC and LSC if one aims at a cure of the disease (Jordan et al. 2006, Visvader 2011, Wang et al. 2005).

Primary tumor cells from patients with AML or MDS are notoriously difficult to maintain ex vivo. Only cells from a fraction of patients can be maintained outside of the patient ex vivo, including in in vitro cell culture systems and xenotransplantation models. In vitro, growth of AML cells can typically only be maintained for several days, and in in vivo transplantation systems, engraftment is typically very low, and many subjects do not engraft at all. Even samples of the patients that do engraft initially, are often not growing aggressively in recipient mouse and remain at a low level, and they also do typically lose their subclonal complexity rapidly, and thus do not reflect well the properties of the primary tumor in the patient, which is greatly limiting the translational relevance of any experimental read-out, e.g. for drug testing.

The present invention addresses the need for models of acute myeloid leukemia and myelodysplastic syndromes for propagating their growth, e.g. for therapeutic target and drug screening testing the effectiveness of treatments.

### SUMMARY OF THE INVENTION

The present invention provides methods for enhancing growth of a human acute myeloid leukemia (AML) sample, a human myelodysplastic syndrome (MDS) sample, a human IL-8 dependent tumor sample, a human preleukemia cell sample, and a human preleukemia clone or subclone ex vivo or in a xenograft animal model, the method comprising adding human interleukin-8 (hIL-8) or a hIL-8 agonist to the sample or administering hIL-8 or a hIL-8 agonist to the animal model or expressing a gene encoding hIL-8 or a hIL-8 agonist in the animal model, wherein hIL-8 or a hIL-8 agonist is present in an amount effective to enhance growth of a human AML sample, a human MDS sample, a human IL-8 dependent tumor sample, a human preleukemia cell sample, or a human preleukemia clone or subclone ex vivo or in a xenograft animal model.

The invention also provides a transgenic animal that expresses a gene encoding human interleukin-8 (hIL-8) or a hIL-8 agonist. This animal model can be used to test the effectiveness of treatments for AML, MDS and IL-8 dependent tumors.

### BRIEF DESCRIPTION OF THE DRAWINGS

FIG. 1A-1C. IL8 is detectable in MDS/AML serum and correlated with disease severity. ELISA for IL8 was done on controls and MDS and AML patient serum samples. Significant increase in IL8 levels were seen in MDS (both low and high risk) and AML samples when compared to controls (P Value<0.05, TTest) (A). IL8 levels were increased after transformation of low risk MDS to higher risk MDS/AML (B) and decreased after treatment with 5-Azacytidine (C).

FIG. 2A-2E. Efficacy of human IL8 addition in patient-derived xenografts from MDS/AML. Treatment of NSG mice with exogenous recombinant human IL8 (rhIL8) leads to higher engraftment from AML sample after 3 weeks of treatment when compared to vehicle controls (representative panel shown in A, B). Injection of recombinant human IL-8 into AML (n=1) or MDS (n=2) xenotransplant recipient mice leads to increased engraftment of primary MDS and AML patients' cells. Averaged data in (C); data from individual subjects in (D). The improvement in engraftment is sustained (as shown for representative PDX) (E).

FIG. 3. Xenotransplantation of primary AML cells into NSG mice pre-transplanted with hIL8-transgenic and doxycycline-induced congenic bone marrow cells leads to a more than 10-fold increase (p=0.0106, N=8) of AML cell engraftment.

### DETAILED DESCRIPTION OF THE INVENTION

The present invention provides a method of enhancing growth of a human acute myeloid leukemia (AML) sample, a human myelodysplastic syndrome (MDS) sample, a human IL-8 dependent tumor sample, a human preleukemia cell sample, or a human preleukemia clone or subclone ex vivo or in a xenograft animal model, the method comprising adding human interleukin-8 (hIL-8) or a hIL-8 agonist to the sample or administering hIL-8 or a hIL-8 agonist to the animal model or expressing a gene encoding hIL-8 or a hIL-8 agonist in the animal model, wherein hIL-8 or the hIL-8 agonist is present in an amount effective to enhance growth of a human AML sample, a human MDS sample, a human IL-8 dependent tumor sample, a human preleukemia cell sample, or a human preleukemia clone or subclone ex vivo or in a xenograft animal model. Preferably, the xenograft animal model is a mouse model. Preferably, the animal is immunocompromised.

Also provided is a method for screening for drugs against human acute myeloid leukemia (AML), human myelodysplastic syndrome (MDS), a human IL-8 dependent tumor, human preleukemia cells, or a human preleukemia clone or subclone, the method comprising contacting the human AML sample, human MDS sample, human IL-8 dependent tumor sample, human preleukemia cell sample, or human preleukemia clone or subclone treated with human interleukin-8 (hIL-8) or a hIL-8 agonist as disclosed herein with a drug ex vivo and determining whether or not the drug reduces growth or differentiation or subclonal complexity, or increases apoptosis, or affects another desired cellular or molecular property of the sample, clone or subclone.

The hIL-8 agonist can be, for example, a synthetic IL-8 agonist. The agonist can comprise a point mutation of hIL-8 or be an hIL-8 derivative that retains the function of hIL-8.

The invention also provides a non-human transgenic animal model (e.g., a xenograft animal model) for engraftment of a human acute myeloid leukemia (AML) sample, a human myelodysplastic syndrome (MDS) sample, a human IL-8 dependent tumor sample, a human preleukemia cell sample, or a human preleukemia clone or subclone, wherein the transgenic animal expresses a gene encoding human interleukin-8 (hIL-8) or a hIL-8 agonist. Preferably, the animal is a mouse. Preferably, the animal is immunocompromised.

Still further provided is a method of making a transgenic mouse model that expresses a gene encoding human interleukin-8 (hIL-8), the method comprising

a) inserting an inducible human IL-8 transgene into an immunocompromised mouse,

b) performing transplantation of hIL-8-transgenic bone marrow cells from the mouse of step a) having the inducible human IL-8 transgene into a parental immunocompromised mouse, and

c) triggering induction of hIL-8 in the mouse of step b) having the transplanted hIL-8-transgenic bone marrow cells,

thereby making a transgenic mouse model that expresses a gene encoding human interleukin-8 (hIL-8).

The inducible human IL-8 transgene can be, for example, a doxycycline-inducible human IL-8 transgene. The transplantation of hIL-8-transgenic bone marrow cells can be, for example, congenic transplantation of hIL-8-transgenic bone marrow cells.

Also provided is a method of making a transgenic mouse model that expresses a gene encoding human interleukin-8 (hIL-8) or a hIL-8 agonist, the method comprising hydrodynamic injection of cDNA encoding hIL-8 or a hIL-8 agonist into an immunocompromised mouse, thereby making a transgenic mouse model that expresses a gene encoding hIL-8 or a hIL-8 agonist.

The immunocompromised mouse can be, for example, a NOD-SCID, NSG or RAG2null mouse.

Also provided is a transgenic mouse model produced by any of the methods disclosed herein.

Preferably, for any of the transgenic animal models disclosed herein, hIL-8 or a h-IL-8 agonist is produced in an amount that is effective to enhance engraftment of human acute myeloid leukemia (AML) cells, human myelodysplastic syndrome (MDS) cells, human IL-8 dependent tumor cells, human preleukemia cells, or a human preleukemia clone or subclone transplanted into the animal model.

Also provided is a method for screening for drugs against human acute myeloid leukemia (AML), human myelodysplastic syndrome (MDS), a human IL-8 dependent tumor, human preleukemia cells, or a human preleukemia clone or subclone, the method comprising administering a drug to any of the animal models disclosed herein and determining whether or not the drug reduces growth or differentiation or subclonal complexity, or increases apoptosis, or affects another desired cellular or molecular property of a human AML, a human MDS, a human IL-8 dependent tumor, human preleukemia cells, or a human preleukemia clone or subclone xenograft in the animal.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

### Introduction

IL8 and its Receptor CXCR2 are Upregulated in MDS and AML Stem Cells and are Indicators of Worse Prognosis in Large Patient Cohorts.

Myeloid malignancies such as MDS and AML can arise from a clone of quiescent cancer-initiating cells that are not eliminated by cytotoxic therapies. HSCs and progenitors in MDS have both quantitative and qualitative alterations at the genetic as well as epigenetic level (Will et al. 2012). Hematopoietic stem cells (HSCs) are expanded in MDS, most significantly in the higher risk subgroups and contain karyotypic abnormalities as well as aberrant epigenetic marks. These results were obtained via establishment of novel protocols to isolate rigorously defined stem and progenitor cell compartments from primary marrow aspirates. Most importantly, karyotypically abnormal MDS HSCs survive even during morphological remissions induced by 5-Azacitidine treatment. Moreover, AML samples with a stem cell like molecular signature have a worse prognosis (Bartholdy et al. 2014) further reinforcing the need to target leukemia stem cells. Taken together, these data along with other studies now demonstrate that these abnormal HSCs need to be targeted for potential curative strategies in MDS/AML.

A transcriptomic analysis of highly purified MDS and AML HSCs and GMPs (n=12) was performed using previously developed assays. IL8 was selectively upregulated by several logfold in these leukemia initiating populations when compared to healthy controls (Schinke et al. 2015). Further validation in another independent cohort showed that the IL8 receptor, CXCR2, was also significantly increased in a cohort of 183 MDS CD34+ samples when compared to 17 healthy controls and was associated with lower hemoglobin and higher transfusion requirements. Higher expression of the IL8 receptor was also seen in the large TCGA AML cohort and was associated with adverse overall survival, further pointing to the critical role of IL8-CXCR2 axis in AML/MDS.

Together, these findings strongly suggested that stimulation through the IL-8-CXCR2 axis is functionally critical for the survival and growth of AML and MDS cells including at the stem cell level.

Inhibition of IL8/CXCR2 Pathway Leads to Decreased Proliferation and Cell Cycle Arrest in Leukemic Cells and Increased Survival in Xenografts.

To determine the functional role of the IL8-CXCR2 pathway in leukemia cells, it was demonstrated that numerous AML cell lines significantly overexpressed CXCR2, when compared to healthy CD34+ cells (Schinke et al. 2015). A specific inhibitor of CXCR2, SB332235, which is a clinically available compound that has 100-fold selectivity for CXCR2 over CXCR121, was used to demonstrate that treatment led to a dose dependent decrease in proliferation in all cell lines, while only minimally affecting growth of healthy control CD34+ cells (Schinke et al. 2015). CXCR2 inhibition also led to significant inhibition of proliferation in primary samples from AML and high risk MDS cases.

shRNAs were designed against CXCR2 (Schinke et al. 2015). Decreased expression of this receptor also led to significantly reduced leukemic colony formation capacity of AML cell lines (p<0.001). Cell cycle analysis was performed to determine the mechanism of growth arrest. A significant arrest of AML cells in the GO stage (p<0.05) was observed after CXCR2 inhibition. At the molecular level, pharmacologic inhibition of CXCR2 led to abrogation of IL8-stimulated signaling in these cells. The efficacy of CXCR2 knockdown was examined in vivo using xenografts with U937 cells. U937 cells were infected with lentiviruses containing shRNA directed against CXCR2 or scrambled control shRNAs, and a fluorescent reporter gene (GFP); cells were sorted for GFP and xenografted into NOD scid gamma (NSG) immunodeficient mice. CXCR2 knockdown led to significant improvement in overall survival (P Value=0.02, Log Rank) (Schinke et al. 2015), demonstrating CXCR2 as a therapeutic target in vivo.

### Results

High IL8 Levels are Detectable in Serum of Patients with MDS and AML.

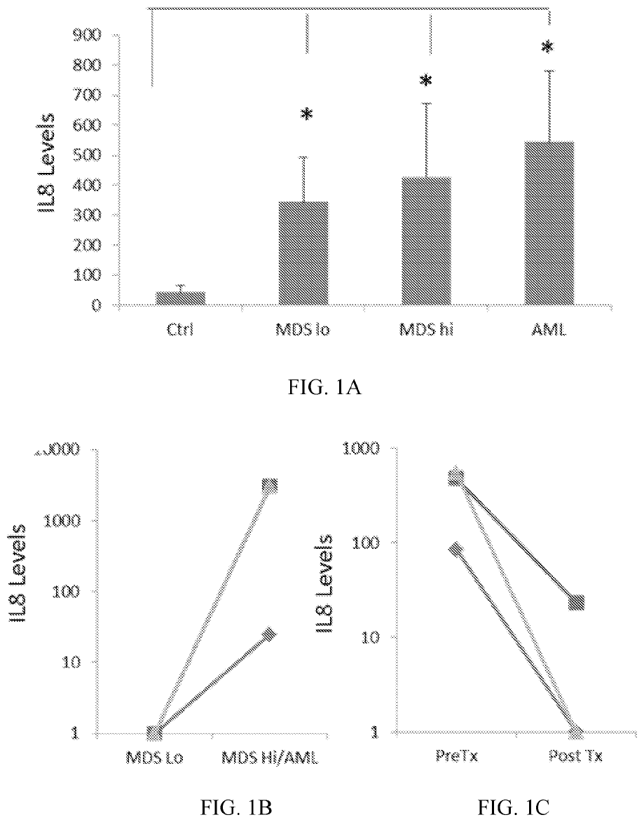

It was previously shown that IL8 mRNA is overexpressed in MDS and AML cells. It was now determined whether IL8 can be detected in serum of patients with MDS and AML. A total of 33 patients (MDS low risk (21), MDS high risk (6) and AML (6)) and 30 controls sera were collected and analyzed for IL8 levels by ELISA. Both MDS and AML samples had significantly elevated levels in serum (FIG. 1A). Furthermore, IL8 levels went up in 3 patients that transformed from low risk MDS to higher risk MDS or AML (FIG. 1B). Additionally, IL8 levels dropped after treatment in another 3 patients that were treated with 5-Azacytidine (FIG. 1C).

Xenograft Models of AML and MDS.

The ex vivo modeling of cells from AML and MDS patients is very challenging. Only cells from ˜60% of patients can be maintained for a short period of time in vitro (˜1-2 weeks) and no long-term culture systems are available for the vast majority of patients (>90%). Only from a very small subset of patients is it possible to derive cell lines that survive for multiple passages. In order to circumvent this challenge, numerous xenotransplantation models have been developed in the past 20 years (for review see Goyama et al. 2015). However, even the most recent models including NOD-SCID IL2-receptor-gamma null mice only allow engraftment of cells from about 50% of AML patients (less than 20% of MDS patients), and even the ones that engraft do so at very low levels of typically about 0.1-1% chimerism in the blood and bone marrow of recipient animals. In addition, subclonal complexity of the primary sample is not maintained upon engraftment, i.e. one or very few sublcones are selected which greatly limits any following experimental readout with regards to its relevance for the real behavior in patients. This has been a major obstacle for research on MDS and AML, for functional studies of human patients' cells, but also for drug testing/development efforts. One challenge of xenograft models is the incomplete compatibility of mouse and human stimulatory factors including cytokines. One approach has been to “humanize” mice through the external or transgenic addition of human cytokines to enhance AML cell engraftment. This strategy has led to some modest success (for review see Goyama et al. 2015) but engraftment numbers and percentages still remain low.

After discovery of IL-8/CXCR2 as a key upregulated pathway in MDS and AML stem cells, we investigated whether an murine homolog/ortholog exists in mice. Through search of literature and sequencing data bases, we found that there is indeed no bona fide murine IL-8 (Cxcl8) gene, and the closest homolog, murine Cxcl15 (which in some data bases is listed as “mouse IL-8”) has only a sequence homology of about 35% with human IL-8. Thus, we hypothesized that xenotransplanted human MDS and AML cells would not have sufficient stimulation through this pathway in recipient mice, and that the addition of human recombinant IL-8 could enhance maintenance and growth of human MDS and AML cells.

IL8 Supplementation In Vivo Leads to Increased Leukemic Engraftment.

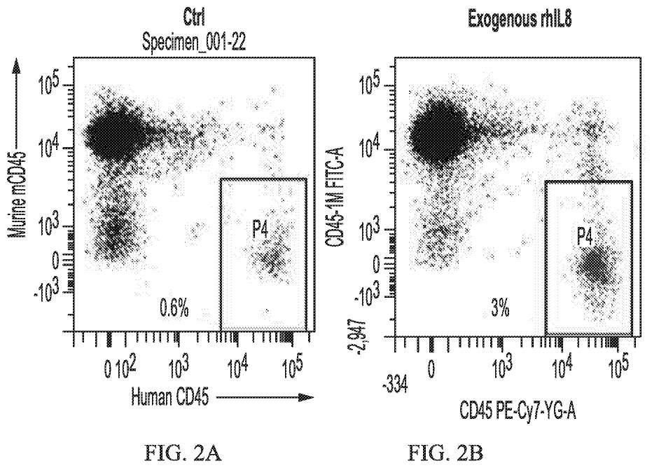

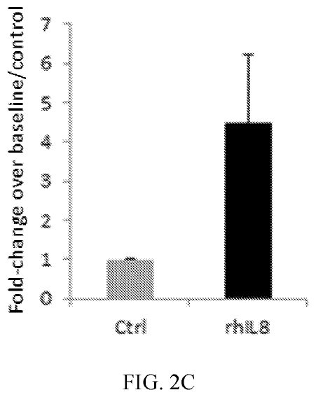

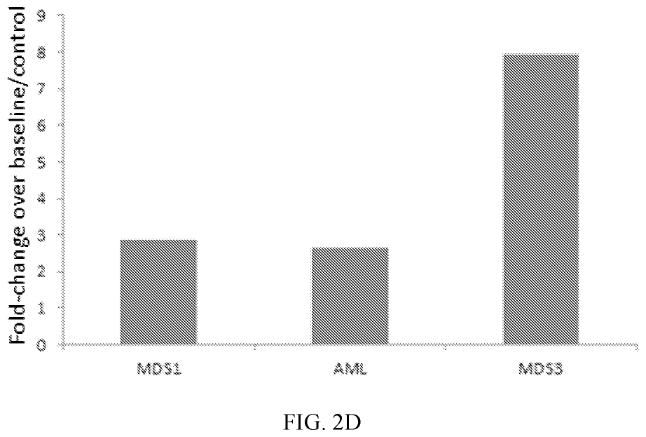

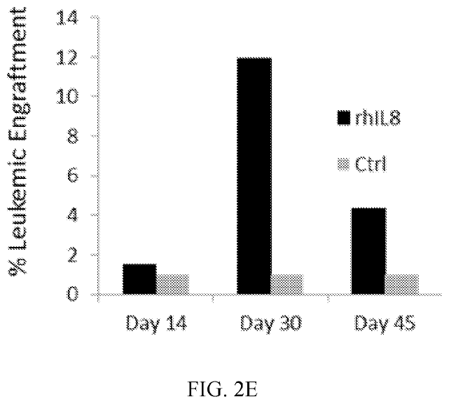

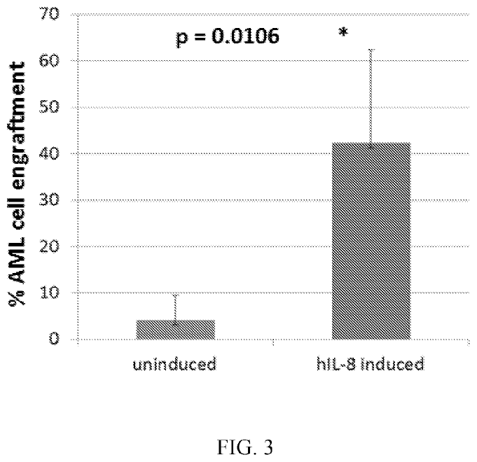

Since our data demonstrated that IL8 is an essential survival factor of MDS and AML cells, we wanted to determine whether exogenous supplementation with human IL8 could lead to better engraftment of human MDS and AML. We evaluated the efficacy of this approach in 3 patient derived xenografts from MDS/AML samples (1 patient with AML, 2 with MDS) and observed a highly significant increase in MDS/leukemic engraftment after IL8 supplementation (FIG. 2C, individual example FIG. 2A-B). These data support the critical role of IL8 in stimulating leukemic growth in vivo and also demonstrate the feasibility of the supplementation increasing xenografting efficiencies in mice.

Exogenous supplementation of recombinant human IL-8 is expensive. However, this finding now opens the door for other strategies, e.g. hydrodynamics-based gene transduction, or the generation of xenograft recipient mice with a human IL-8 transgene. Such systems will allow the more efficient xenografting of MDS and AML patients' samples and may thus enable for the first time the high-throughput screening of compounds or genetic reagents in primary human MDS and AML.

Generation of a Human IL-8 Transgenic Mouse in the NSG Background, and In Vivo Xenotransplantation Data in this Model.

We have generated a new transgenic mouse model in which we inserted a doxycycline-inducible human IL-8 transgene into the immunocompromised NSG mouse strain. We then performed congenic transplantation of hIL-8-transgenic bone marrow cells into 8 parental NSG mice, followed by doxycycline-triggered induction of hIL-8 in 4 of the recipient mice. 4 uninduced mice served as a control. We then xenotransplanted 5×10⁶ leukemic cells from an AML patient into these animals and determined AML cell engraftment in the bone marrow 4 months post-transplantation. AML cells are notoriously difficult to transplant and in most cases are not suitable for expansion and use in patient derived xenograft (PDX) models due to low engraftment (here: control showed very low engraftment of only 4% ion average). The transplanted recipients with induced hIL-8 showed strikingly higher engraftment of primary AML patient cells (average: 42.2%, 10.4-fold increase, p=0.0106, N=8). These data demonstrate that human IL-8 is indeed critical for the engraftment and maintenance of human AML stem cells (AML-initiating cells) in vivo. We have now a novel transgenic mouse system available that permits for the first time the generation of robust primary xenografts and PDX models from AML and MDS patients' cells, which can be used for drug development and testing purposes as well as functional/mechanistic studies.

### REFERENCES

Bartholdy B, Christopeit M, Will B, Mo Y, Barreyro L, Yu Y, Bhagat T D, Okoye-Okafor U C, Todorova T I, Greally J M, Levine R L, Melnick A, Verma A, Steidl U. HSC commitment-associated epigenetic signature is prognostic in acute myeloid leukemia. J Clin Invest. 2014 March; 124(3):1158-67.
Goyama S, Wunderlich M, Mulloy J C. Xenograft models for normal and malignant stem cells. Blood. 2015 Apr. 23; 125(17):2630-40. doi: 10.1182/blood-2014-11-570218. Epub 2015 Mar. 11.
Hanahan D, Weinberg R A: The hallmarks of cancer. Cell 2000, 100(1):57-70.
Jordan C T, Guzman M L, Noble M: Cancer stem cells. N Engl J Med 2006, 355(12):1253-1261.
Schinke C, Giricz O, Li W, Shastri A, Gordon S, Barreyro L, Bhagat T, Bhattacharyya S, Ramachandra N, Bartenstein M, Pellagatti A, Boultwood J, Wickrema A, Yu Y, Will B, Wei S, Steidl U, Verma A. IL8-CXCR2 pathway inhibition as a therapeutic strategy against MDS and AML stem cells. Blood. 2015 May 14; 125(20):3144-52. doi: 10.1182/blood-2015-01-621631. Epub 2015 Mar. 25.
Visvader J E: Cells of origin in cancer. Nature 2011, 469(7330):314-322.
Wang J C, Dick J E: Cancer stem cells: lessons from leukemia. Trends Cell Biol 2005, 15(9):494-501.
Will B, Zhou L, Vogler T O, Ben-Neriah S, Schinke C, Tamari R, Yu Y, Bhagat T D, Bhattacharyya S, Barreyro L, Heuck C, Mo Y, Parekh S, McMahon C, Pellagatti A, Boultwood J, Montagna C, Silverman L, Maciejewski J, Greally J M, Ye B H, List A F, Steidl C, Steidl U, Verma A. Stem and progenitor cells in myelodysplastic syndromes show aberrant stage-specific expansion and harbor genetic and epigenetic alterations. Blood. 2012 Sep. 6; 120(10):2076-86. Epub 2012 Jul. 2.

1. A method of enhancing growth of a human acute myeloid leukemia (AML) sample, a human myelodysplastic syndrome (MDS) sample, a human IL-8 dependent tumor sample, a human preleukemia cell sample, or a human preleukemia clone or subclone ex vivo or in a xenograft animal model, the method comprising adding human interleukin-8 (hIL-8) or a hIL-8 agonist to the sample or administering hIL-8 or a hIL-8 agonist to the animal model or expressing a gene encoding hIL-8 or a hIL-8 agonist in the animal model, wherein hIL-8 or the hIL-8 agonist is present in an amount effective to enhance growth of a human AML sample, a human MDS sample, a human IL-8 dependent tumor sample, a human preleukemia cell sample, or a human preleukemia clone or subclone ex vivo or in a xenograft animal model.

2. The method of claim 1, wherein the xenograft animal model is a mouse model.

3. The method of claim 1, wherein the animal is immunocompromised.

4. A non-human transgenic animal model for engraftment of a human acute myeloid leukemia (AML) sample, a human myelodysplastic syndrome (MDS) sample, a human IL-8 dependent tumor sample, a human preleukemia cell sample, or a human preleukemia clone or subclone, wherein the transgenic animal expresses a gene encoding human interleukin-8 (hIL-8) or a hIL-8 agonist.

5. The transgenic animal model of claim 4, wherein the animal is a mouse.

6. The animal of claim 4, wherein the animal is immunocompromised.

7. A method of making a transgenic mouse model that expresses a gene encoding human interleukin-8 (hIL-8), the method comprising
a) inserting an inducible human IL-8 transgene into an immunocompromised mouse,
b) performing transplantation of hIL-8-transgenic bone marrow cells from the mouse of step a) having the inducible human IL-8 transgene into a parental immunocompromised mouse, and
c) triggering induction of hIL-8 in the mouse of step b) having the transplanted hIL-8-transgenic bone marrow cells,
thereby making a transgenic mouse model that expresses a gene encoding human interleukin-8 (hIL-8).

8. The method of claim 7, wherein the inducible human IL-8 transgene is a doxycycline-inducible human IL-8 transgene.

9. The method of claim 7, wherein the transplantation of hIL-8-transgenic bone marrow cells is congenic transplantation of hIL-8-transgenic bone marrow cells.

10. A method of making a transgenic mouse model that expresses a gene encoding human interleukin-8 (hIL-8) or a hIL-8 agonist, the method comprising hydrodynamic injection of cDNA encoding hIL-8 or a hIL-8 agonist into an immunocompromised mouse, thereby making a transgenic mouse model that expresses a gene encoding hIL-8 or a hIL-8 agonist.

11. The method of claim 7, wherein the immunocompromised mouse is a NOD-SCID, NSG or RAG2null mouse.

12. A transgenic mouse model that expresses a gene encoding human interleukin-8 (hIL-8) or a hIL-8 agonist produced by the method of claim 7.

13. The transgenic animal model of claim 1, wherein hIL-8 or a hIL-8 agonist is produced in an amount that is effective to enhance engraftment of human acute myeloid leukemia (AML) cells, human myelodysplastic syndrome (MDS) cells, human IL-8 dependent tumor cells, human preleukemia cells, or a human preleukemia clone or subclone transplanted into the animal model.

14. A method for screening for drugs against human acute myeloid leukemia (AML), human myelodysplastic syndrome (MDS), a human IL-8 dependent tumor, human preleukemia cells, or a human preleukemia clone or subclone, the method comprising contacting the human AML sample, human MDS sample, human IL-8 dependent tumor sample, human preleukemia cell sample, or human preleukemia clone or subclone of claim 1 with a drug ex vivo and determining whether or not the drug reduces growth or differentiation or subclonal complexity or increases apoptosis of the sample, clone or subclone.

15. A method for screening for drugs against human acute myeloid leukemia (AML), human myelodysplastic syndrome (MDS), a human IL-8 dependent tumor, human preleukemia cells, or a human preleukemia clone or subclone, the method comprising administering a drug to the animal model of claim 1 and determining whether or not the drug reduces growth or differentiation or subclonal complexity or increases apoptosis of a human AML, a human MDS, a human IL-8 dependent tumor, human preleukemia cells, or a human preleukemia clone or subclone xenograft in the animal.
